(19) United States
(12) Patent Application Publication
(10) Pub. No.: US 2008/0081370 A1
(43) Pub. Date: Apr. 3, 2008

(54) DIRECTED DIFFERENTIATION OF HUMAN EMBRYONIC STEM CELLS INTO MESENCHYMAL/STROMAL CELLS

(76) Inventors: Peiman Hematti, Middleton, WI (US); Parul A. Trivedi, Madison, WI (US)

Correspondence Address: QUARLES & BRADY LLP, 33 E. MAIN ST, SUITE 900, P.O. BOX 2113, MADISON, WI, 53701-2113, US

(21) Appl. No.: 11/864,538

(22) Filed: Sep. 28, 2007

Related U.S. Application Data
(60) Provisional application No. 60/847,713, filed on Sep. 28, 2006.

(51) Int. Cl. C12N 5/08; C12N 5/02

(52) U.S. Cl. 435/372; 435/395; 435/402

(57) ABSTRACT

Methods for producing hESC-derived mesenchymal stromal/stem cells are disclosed. The cells produced are multipotent and can differentiate into the three mesenchymal cell lineage types and are characterized by cell-specific markers.

### CROSS-REFERENCE TO RELATED APPLICATIONS

This application claims a priority benefit to U.S. Provisional Patent Application No. 60/847,713, filed Sep. 28, 2006, incorporated herein by reference as if set forth in its entirety.

### STATEMENT REGARDING FEDERALLY SPONSORED RESEARCH OR DEVELOPMENT

This invention was made with United States government support awarded by the following agency: NIH CA014520. The United States government has certain rights in this invention.

### BACKGROUND

The invention relates generally to methods for culturing human embryonic stem cells (hESCs), and more particularly to methods for differentiating cultured hESCs into mesenchymal stromal/stem cells (MSCs).

MSCs can differentiate into at least three downstream mesenchymal lineages (i.e., osteoblasts, chondrocytes and adipocytes). No unique, characteristic MSC marker exists, so morphological, immunophenotypical and functional criteria are used to identify such cells. See, Horwitz E, et al., “Clarification of the nomenclature for MSC: The International Society for Cellular Therapy position statement,” Cytotherapy 7:393 (2005); and Dominici M, et al., “Minimal criteria for defining multipotent mesenchymal stromal cells. The International Society for Cellular Therapy position statement,” Cytotherapy 8:315 (2006), each of which is incorporated herein by reference as if set forth in its entirety. Because MSCs can differentiate into many cell types, the art contemplates methods for differentiating MSCs for cell-based therapies, for regenerative medicine and for reconstructive medicine.

MSCs have been isolated from such tissues as adult bone marrow, fat, cartilage and muscle. Pittenger F, et al., “Multilineage potential of adult human mesenchymal stem cells,” Science 284:143-147 (1999); Zuk P, et al., “Multilineage cells from human adipose tissue: implications for cell-based therapies,” Tissue Eng. 7:211-228 (2001); and Young H, et al., “Human reserve pluripotent mesenchymal stem cells are present in the connective tissues of skeletal muscle and dermis derived from fetal, adult, and geriatric donors,” Anat. Rec. 264:51-62 (2001), each of which is incorporated herein by reference as if set forth in its entirety. MSCs have also been isolated from human peripheral blood. Kassis I, et al., “Isolation of mesenchymal stem cells from G-CSF-mobilized human peripheral blood using fibrin microbeads,” Bone Marrow Transplant. 37:967-976 (2006), incorporated herein by reference as if set forth in its entirety. Likewise, MSCs have been isolated from human neonatal tissue, such as Wharton's jelly (Wang H, et al., “Mesenchymal stem cells in the Wharton's jelly of the human umbilical cord,” Stem Cells 22:1330-1337 (2004)); human placenta (Fukuchi Y, et al., “Human placenta-derived cells have mesenchymal stem/progenitor cell potential,” Stem Cells 22:649-658 (2004)); and umbilical cord blood (Erices A, et al., “Mesenchymal progenitor cells in human umbilical cord blood,” Br. J. Haematol. 109:235-242 (2000)). Furthermore, MSCs have been isolated from human fetal tissues. Campagnoli C, et al., “Identification of mesenchymal stem/progenitor cells in human first-trimester fetal blood, liver, and bone marrow,” Blood 98:2396-2402 (2001).

The art is limited, however, by an inability to isolate a sufficient number of MSCs for subsequent differentiation and use. Where suitable donors are available, procedures required to isolate even a limited number of cells are invasive, and thus harbor risks to donors. Such donor MSCs need to be tested extensively to make sure that are free of any transmittable infectious agents. It also remains difficult to maintain isolated MSCs in long-term culture, as well as to maintain such cultures free of bacterial or viral contamination.

To address the inability to isolate a sufficient number of MSCs, researchers investigated methods for differentiating hESCs to MSC. Unfortunately, efforts to date either require culture for a substantial time on potentially contaminating feeder layers, utilize methods that might not be clinically applicable, or yield cells that retain characteristics of undifferentiated hESCs. For example, Barberi et al. differentiated hESCs on mitotically inactivated mouse stromal cell lines (i.e., feeder cells) with 20% heat-inactivated fetal bovine serum (FBS) in α-MEM medium for 40 days. Barberi T, et al. “Derivation of multipotent mesenchymal precursors from human embryonic stem cells,” PLoS Med. 2:e161 (2005), incorporated herein by reference as if set forth in its entirety. Cells were harvested and assayed for CD73; sorted CD73⁺ cells were then plated in the absence of the stromal feeder cells with 20% FBS in α-MEM for 7 to 10 days. Barberi et al. differentiated the MSC into adipogenic cells, chondrogenic cells, osteogenic cells and myogenic cells.

Additionally, Olivier et al. differentiated hESCs into MSCs by plating raclures (i.e., spontaneously differentiated cells that appear in hESC culture in the center or at the edges of colonies) with D10 medium (DMEM, 10% FBS, 1% penicillin/streptomycin and 1% non-essential amino acids) changed weekly until a thick multi-layer epithelium developed. Olivier E, et al., “Differentiation of human embryonic stem cells into bipotent mesenchymal stem cells,” Stem Cells 24:1914-1922 (2006), incorporated herein by reference as if set forth in its entirety. After approximately four weeks, MSCs were isolated by dissociation of the epithelium with a mixture of trypsin, collagenase type IV and dispase for four to six hours, followed by re-plating in D10 medium. Olivier et al.'s MSCs grew robustly, had stable karyotypes, were contact inhibited, senesced after twenty passages and differentiated into adipogenic cells and osteogenic cells. Olivier et al. did not report that the cells could differentiate into chondroblasts. Unlike Barberi et al., Olivier et al. did not require a feeder layer to support differentiation of hESC into MSCs. However, Olivier et al.'s MSCs tested SSEA-4 positive, suggesting that these MSCs still carry cell surface markers characteristic of hESC.

Furthermore, Pike & Shevde differentiated hESCs to form a population of MSCs via embryoid body intermediates incubated for ten to twelve days in a mesenchymal-specific medium (MesenCult® with 10% FBS; α-MEM with glutamine and nucleosides; or DMEM with glucose and glutamine, replaced every two days). US Patent Publication No. 2006/0008902, incorporated herein by reference as if set forth in its entirety. The incubated embryoid body intermediates were digested, and resulting pre-mesenchymal cells were cultured to 80% confluence. The cells were trypsinized and passaged three times in mesenchymal-specific medium. However, Pike & Shevde's MSCs tested Oct-4 positive, suggesting that these MSCs still carry cell surface markers characteristic of hESC.

For the foregoing reasons, there is a continuing need for sources of MSCs, especially MSCs derived from hESCs in vitro.

### BRIEF SUMMARY

The present invention relates to methods for differentiating hESCs to an essentially homogenous population of MSCs. As used herein, “essentially homogenous” means that at least 95% of the population expresses CD73. One can use the hESC-derived MSCs thus obtained, or cell types obtained by further differentiating the MSCs in any research and clinical application in which MSCs or derivative cell types obtained from any other source can be used.

In a first aspect, the present invention is summarized as a method for making MSCs in vitro that includes passaging hESCs in a culture on a basement membrane matrix surface in a mouse, feeder-cell, conditioned medium containing basic fibroblast growth factor (MEF-CM/bFGF). The culture is substantially free of feeder cells, and the medium is renewed no more frequently than every third day and no less frequently than every fifth day until at least about 40%, or at least about 50%, or at least about 60% of the passaged cells have a spindle-shaped, stromal/fibroblast differentiated cell morphology. At this point, cells lacking the differentiated cell morphology may be optionally removed from the culture. The method also includes passaging and culturing cells having the differentiated cell morphology on a basement membrane matrix surface in MEF-CM/bFGF, and splitting cells having the differentiated cell morphology under the same conditions when the cells are at least about 60% confluent. Again, the medium is renewed no more frequently than every third day and no less frequently than every fifth day until the plates were about 80% (total cells) confluent. The method further includes passaging and culturing the differentiated cells until confluent in a medium that supports growth of MSCs until the cells are >95% confluent. Optionally, the cells may be cultured on a plastic, gelatin-coated surface. The resulting cells have a MSC-characteristic spindle-shaped morphology and express MSC surface markers (i.e., CD73+, CD29+, CD44+, CD90+, CD105+, but are Oct-4−, CD34− and CD45−). Again, cells that lack the differentiated cell morphology may be optionally removed from the culture.

In a second aspect, the present invention is summarized as a method for making MSCs in vitro that includes passaging hESCs in a culture on a basement membrane matrix surface in MEF-CM/bFGF. The culture is substantially free of feeder cells, and the medium is renewed no more frequently than every third day and no less frequently than every fifth day until at least about 40%, or at least about 50%, or at least about 60% of the passaged cells have a spindle-shaped, stromal/fibroblast differentiated cell morphology (i.e., spindle-shaped). The method also includes passaging and culturing the differentiated cells until confluent on a plastic surface that is optionally gelatin-coated in a medium that supports growth of MSCs. Again, the medium is renewed no more frequently than every third day and no less frequently than every fifth day until about 80%-85% confluence (and most of the cells expressing CD73). The resulting cells having a MSC-characteristic spindle-shaped morphology and expressing MSC cell markers.

In a third aspect, the present invention is summarized as a method for making MSCs in vitro that includes passaging hESCs in a culture on a basement membrane matrix surface in a complete, serum-free medium. The medium is renewed no more frequently than every third day and no less frequently than every fifth day until at least about 40%, or at least about 50%, or at least about 60% of the passaged cells have a spindle-shaped, stromal/fibroblast differentiated cell morphology. The method also includes passaging and culturing the differentiated cells until confluent on a plastic surface that is optionally gelatin-coated in a medium that supports growth of MSCs. The medium is renewed no more frequently than every third day and no less frequently than every fifth day until about 80%-85% confluence (and most of the cells expressing CD73). The resulting cells having a MSC-characteristic spindle-shaped morphology and expressing MSC cell markers.

In some embodiments of any aspect, at least about 60%, at least about 75%, at least about 85%, or at least about 95% of the cells maintained on the plastic surface (which may be gelatin-coated) are MSCs, as characterized by cell surface markers, particularly CD73. Advantageously, the MSCs produced in the method are SSEA-4-negative and Oct-4 negative.

In some embodiments of any aspect, bFGF is at a concentration between 3-8 ng/ml.

These in vitro differentiation methods have many advantages over existing methods for obtaining MSCs. For example, the methods avoid invasive harvesting procedures and avoid the need to purify cells from human tissue. In addition, the methods avoid the need to culture hESCs on an animal cell line and/or avoid the need to produce embryoid bodies as an intermediate product. Furthermore, the methods produce large and substantially homogenous populations of karyotypically normal MSCs that, inter alia, can be used as xenogenic-free, pathogen-free feeder cells in cultures of hESC or other cells. The MSCs can also be differentiated to produce cells in the mesenchymal cell lineages for clinical applications or can be manipulated using the tools of molecular biology to facilitate study of the molecular basis of MSC multipotentcy and differentiation. Moreover, the MSCs have cell surface markers, differentiation potentials (i.e., can differentiate in to osteoblasts, adipocytes and chondrocytes) and immunological properties that are similar, at least in vitro, to MSCs derived from adult bone marrow. Likewise, the MSCs adhere to plastic in standard culture conditions.

These and other features, aspects and advantages of the present invention will be more fully understood from the description that follows. The description of preferred embodiments is not intended to limit the invention to cover all modifications, equivalents and alternatives. Reference should therefore be made to the claims herein for interpreting the scope of the invention.

### BRIEF DESCRIPTION OF THE DRAWINGS

Not applicable.

### DESCRIPTION OF PREFERRED EMBODIMENTS

The present invention relates to the inventors' observation that frequency of medium changes affects differentiation of hESCs to MSCs. That is, daily medium changes or medium changes every other day maintain the hESCs in an undifferentiated state; whereas medium changes every six to seven days differentiate hESCs into other cell types. This observation suggests that hESCs differentiate to MSCs more efficiently and to a greater extent with medium changes every three to five days.

Briefly, hESCs are cultured on a basement membrane matrix surface (e.g. Matrigel®, laminin, collagen, glycosaminoglycans, osteocalcin, osteonectin and mixtures thereof) in MEF-CM/bFGF. The culture is substantially free of feeder cells, and the medium is renewed no more frequently than every third day and no less frequently than every fifth day until at least about 40%, or at least about 50%, or at least about 60% of the passaged cells have a stromal/fibroblast differentiated cell morphology (i.e., spindle shaped). Optionally, undifferentiated cells are removed by scraping or aspiration from the culture. Cells having the differentiated cell morphology are passaged with collagenase and cultured on a basement membrane matrix surface in MEF-CM/bFGF, and split under the same conditions when the cells are at least about 60% confluent. Differentiated cells are then passaged with trypsin and cultured until confluent on a plastic, gelatin-coated surface (0.1%-0.5%) in a medium that supports growth of MSCs (i.e., DMEM+10% FBS; MesenCult® (StemCell Technologies; Vancouver, Canada); or MesenPro RS™ (Invitrogen; Carlsbad, Calif.)), so that the resulting cells have a MSC-characteristic spindle-shaped morphology and express MSC cell markers.

Alternatively, hESCs are cultured on a basement membrane matrix surface (e.g., Matrigel®, laminin, collagen, glycosaminoglycans, osteocalcin, osteonectin and mixtures thereof) in MEF-CM/bFGF. The culture is substantially free of feeder cells, and the medium is renewed no more frequently than every third day and no less frequently than every fifth day until at least about 40%, or at least about 50%, or at least about 60% of the passaged cells have a spindle-shaped stromal/fibroblast differentiated cell morphology. Cells having the differentiated cell morphology are passaged with trypsin onto a plastic surface that may be gelatin-coated in a medium that supports growth of MSCs, and split under the same conditions when the cells are at least about 60% confluent. The resulting cells have a MSC-characteristic spindle-shaped morphology and express MSC cell markers. Cells that lack the differentiated cell morphology are not removed from the culture. MSCs grow on non-gelatin coated plates, but gelatin-coated plates provide robust (fast) growth of MSCs.

Alternatively, hESCs are cultured on a basement membrane matrix surface (e.g., Matrigel®, laminin, collagen, glycosaminoglycans, osteocalcin, osteonectin and mixtures thereof) in a complete, serum-free medium (e.g., mTeSR™). See, Ludwig T, et al., “Derivation of human embryonic stem cells in defined conditions,” Nat. Biotechnol. 24: 185-187 (2006); and Ludwig T, et al., “Feeder-independent culture of human embryonic stem cells,” Nat. Methods 3:637-646 (2006), each of which is incorporated herein by reference as if set forth in its entirety. Consequently, the culture is free of feeder cells, conditioned medium and mouse embryonic feeder (MEF) cells. The medium is renewed no more frequently than every third day and no less frequently than every fifth day until at least about 40%, or at least about 50%, or at least about 60% of the passaged cells have a spindle-shaped stromal/fibroblast differentiated cell morphology. Cells having the differentiated cell morphology are passaged with trypsin onto a plastic surface that may be gelatin-coated in a medium that supports growth of MSCs, and split under the same conditions when the cells are at least about 60% confluent. The resulting cells have a MSC-characteristic morphology and express MSC cell markers. Cells that lack the differentiated cell morphology are not removed from the culture. Again, MSCs grow on non-gelatin coated plates, but gelatin-coated plates provide robust (fast) growth of MSCs.

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are now described.

As used herein, stromal/fibroblast cell morphology means that a cell has a spindle-shaped morphology (i.e., the cells have a long elongated shape as opposed to a round shaped characteristic of hESCs).

The invention will be more fully understood upon consideration of the following non-limiting Examples.

### EXAMPLES

### Example 1

### Direct Differentiation of hESCs into MSCs

H1, H7 and H9 hESC cells lines from WiCell Research Institute, Inc. (Madison, Wis.) were cultured and maintained as directed on mouse embryonic fibroblast (MEF) feeder layers. hESC colonies were then transferred onto Matrigel®-coated plates (BD Biosciences; San Jose, Calif.) and were cultured in MEF-CM/bFGF (containing between 4 ng/ml to 8 ng/ml of bFGF; Invitrogen). MEF-CM medium is hESC medium with 4 ng/ml of bFGF added on MEF irradiated with 50-60 Gy, which is collected every 24 hours for 7 days. Another 4 ng/ml of bFGF was added to the above MEF-CM and filtered before use for maintenance of hESCs on Matrigel®-coated plates. The medium was changed daily until no evidence of MEF feeder cells remained in the culture. Undifferentiated hESCs were passaged onto new Matrigel®-coated plates whenever the cells became confluent.

For differentiation, the hESCs were then passaged onto new Matrigel®-coated plates with MEF-CM/bFGF, and the medium was changed every third to fifth day. Under these culture conditions, an increasing percentage of the cells displayed a spindle-shaped, stromal/fibroblast cell morphology after two to three passages. Thus, differentiation of hESCs toward MSCs was influenced by the interval of medium culture changes.

After 7 to 10 days, about 40% to about 50% of cells on confluent culture plates had the stromal/fibroblast cell morphology. Cells at the periphery of the hESC colonies and/or between two hESC colonies were the first to show the stromal/fibroblast cell morphology. Remaining undifferentiated or semi-differentiated hESCs were removed from the plate by scraping and aspiration, which is an optional, but preferred, step.

At confluence, remaining cells were split using collagenase (1 mg/ml; Invitrogen) onto new Matrigel®-coated plates with MEF-CM/bFGF. Medium changes continued every three to five days. When the majority of cells showed stromal/fibroblast cell morphology (about two to four medium changes), remaining undifferentiated or semi-differentiated hESC colonies were removed as described above.

Cell having the stromal/fibroblast cell morphology were then passaged using trypsin (0.05%; Invitrogen) onto 0.1% to 0.5% gelatin-coated plates (Type A gelatin; Sigma; St. Louis, Mo.) in MSC medium (α-MEM, 10% FBS, 0.1 mM non-essential amino acids and 2 mM L-glutamine). Cells were more than 90% positive for CD73 (a MSC marker) and about 1% to about 2% positive for SSEA-4 (a hESC marker), and essentially all of the cells had the stromal/fibroblast cell morphology. Any further passages were performed using trypsin, gelatin-coated plates and MSC medium. In any higher passages, cells uniformly expressed MSC markers such as CD73, but were SSEA-4 negative. Cells were maintained continuously on MSC medium for over three months and maintained their ability to differentiate. As such, MSCs were consistently obtained in four to six weeks with this method.

hESC-derived MSCs showed fibroblastic/spindle morphology and were karyotypically normal when tested at passage 4 or 5. These cells were also frozen, thawed and subsequently passaged. Likewise, the hESC-derived MSCs were passaged between 18-21 passages before they started to show signs of senescence, or slowed growth.

hESC-derived MSCs possessed cell surface markers characteristic of bone marrow-derived MSCs (BM-MSCs). Cells were assayed by fluorescent activated cell sorting (FACS) for cell surface markets by flow cytometry, the results of which are shown in Table 1. For FACS, single cell suspensions were analyzed using a FACSCalibur™ flow cytometer with CellQuest™ acquisition software (BD Biosciences) and FlowJo software (Tree Star; Ashland, Oreg.). Only human-specific monoclonal antibodies were used and were obtained from BD Biosciences, eBioscience (San Diego, Calif.) or R&D Systems (Minneapolis, Minn.). The cells were compared to BM-MSCs (e.g., BM-MSC-1215 and BM-MSC-5066R) that served as control and exhibited similar cell surface marker characteristics.

TABLE 1
Cell surface markers assayed for on hESC-derived MSC and BM-MSCs.

| | H1-derived | H7-derived | H9-derived | BM-derived | BM-derived |
|---|---|---|---|---|---|
| Marker | MSCs | MSCs | MSCs | MSCs (1215) | MSCs (5066R) |
| CD29 | 99.5% | 99.8% | 98.3% | 98.6% | 96.8% |
| CD31 | Negative | Negative | Negative | Negative | Negative |
| CD34 | Negative | Negative | Negative | Negative | Negative |
| CD38 | Negative | Negative | Negative | Negative | Negative |
| CD40 | Negative | Negative | Negative | Negative | Negative |
| CD44 | 98.7% | 97.6% | 97.2% | 97.1% | 97.9% |
| CD45 | Negative | Negative | Negative | Negative | Negative |
| CD54 | 99.1% | 86.3% | 82.6% | 86.0% | 98.1% |
| CD73 | 98.5% | 99.8% | 98.6% | 96.5% | 97.9% |
| CD80 | 1.1% | 1.8% | 5.5% | 3.2% | 9.1% |
| CD90 | 96.9% | 92.3% | 94.2% | 98.2% | 99.9% |
| CD105 | 96.2% | 98.3% | 96.9% | 99.2% | 99.9% |
| HLA-ABC | 97.3 | 99.2%9 | 98.5 | 98.5 | 97.9 |
| HLA-DR | Negative | Negative | Negative | Negative | Negative |
| SSEA-4 | Negative | Negative | Negative | Negative | Negative |

hESC-derived MSCs did not change expression of HLA-ABC, but increased expression of HLA-DR in response to interferon-gamma (IFN-γ). In addition, hESC-derived MSCs did not show significant changes in expression of CD40 and CD80 in response to IFN-γ. BM-MSCs showed similar responses to IFN-Y. Briefly, cell surface marker expression was examined by adding 100 U/ml of IFN-γ (R&D Systems) to the culture medium and analyzing the cells after 1, 3 and 5 days.

Likewise, hESCs-derived MSCs when cultured with peripheral blood mononuclear cells did not elicit a T-cell response regardless of whether MSCs were previously treated with IFN-γ. Briefly, to test the effect of in vitro T-cell response towards MSCs, 5×10⁵ hESC-derived MSCs (or BM-MSCs) that were irradiated (100 Gy) and plated into 24-well plates containing 5×10⁵ carboxyfluorescein diacetate succinimidyl ester (CFSE)-labeled human peripheral blood mononuclear cells in RPM1-1640+10% FBS. After 3 days, cells were harvested and washed twice with PBS+1% FBS. T-cells were labeled with CD3-APC (eBioscience) for 30 minutes at 4° C., washed and re-suspended in PBS+1% FBS. Propidium iodide was added to the samples to exclude dead cells from analysis. Alternatively, MSCs were treated with IFN-γ (100 ng/ml) for 3 days prior to plating with peripheral blood mononuclear cells. Cells were analyzed using a FACSCalibur™ flow cytometer with CellQuest™ acquisition software and FlowJo software.

To test the effect of MSCs on response of responder T-cells to effector T-cells in mixed lymphocyte culture reactions, 5×10⁵ hESC-derived MSCs were added to mixed lymphocyte cultures containing 2.5×10⁵ CFSE-labeled T-lymphocytes (responder cells) and 2.5×10⁵ third party pooled (3-5 donors) peripheral blood mononuclear cells (effector cells). hESC-derived MSCs when added to these mixed lymphocyte cultures suppressed the proliferation of responder T-cells.

### Example 2

### Modified Method of Directed Differentiation of hESCs into MSCs

Methods: H1, H7 and H9 hESCs from WiCell Research Institute, Inc. were used to derive MSCs. These cells were cultured and maintained on a basement membrane matrix surface (e.g., Matrigel®, laminin, collagen, glycosaminoglycans, osteocalcin, osteonectin and mixtures thereof) in MEF-CM/bFGF (containing 8 ng/ml of bFGF). As such, the hESCs were free of MEFs, as described above, which was verified for an absence of mouse HPRT gene using primers specific for this gene by RT-PCR. Likewise, the cells were verified for a presence of human β-actin gene using specific primers for human β-actin gene. Cells were also checked via flow cytometry using SSEA-4-APC and CD73-PE surface antibodies to check that the starting population of cells were hESCs.

For differentiation, the hESCs were passaged onto new Matrigel®-coated plates with MEF-CM/bFGF medium, which was changed every three to five days. Under these culture conditions, an increasing percentage of cells displayed a spindle-shaped, stromal/fibroblast cell morphology after two to three passages. Thus, differentiation of hESCs toward MSCs was influenced by the interval of medium culture changes.

After 7 to 10 days, about 40% to about 50% of cells on confluent culture plates had the stromal/fibroblast cell morphology. Cells at the periphery of the hESC colonies and/or between two hESC colonies were the first to show the stromal/fibroblast cell morphology. Undifferentiated or semi-differentiated hESCs, however, were not removed from the plate by scraping and aspiration, as undifferentiated hESCS did not adhere to plastic plates used below.

At confluence (80% confluent), cells were passaged using trypsin onto either 0.1% gelatin-coated plates or plates without gelatin with MSC medium (α-MEM, 10% FBS, 0.1 mM non-essential amino acids and 2 mM L-glutamine), which was changed every three to five days. Cells were checked for adherence of at least 50-75% of the passaged cells to new plastic plates. Cells were more than 90% positive for CD73 (a MSC marker) and about 1% to about 2% positive for SSEA-4 (a hESC marker), and essentially all of the cells had the stromal/fibroblast cell morphology. Any further passages were performed using trypsin, gelatin-coated (or not) plates and MSC medium. In any higher passages, cells uniformly expressed MSC markers such as CD73 (typically >99% positive), and were SSEA-4 negative. Cells were maintained continuously on MSC medium for over three months and maintained their ability to differentiate, as did cells passaged after freezing and thawing. As such, hESC-derived MSCs were consistently obtained in three to four weeks with this method.

The hESC-derived MSCs were assayed by FACS, as described above, the results of which are shown in Table 2.

TABLE 2
Cell surface markers of hESC-derived MSCs.

| | MSCs on gelatin- | MSCs on non-gelatin- |
|---|---|---|
| Marker | coated plates | coated plates |
| CD29 | 99% | 99.9% |
| CD31 | Negative | Negative |
| CD34 | Negative | Negative |
| CD38 | Negative | Negative |
| CD44 | 99.9% | 99.9% |
| CD45 | Negative | Negative |
| CD54 | 82.9% | 95.2% |
| CD73 | 99% | 99% |
| CD90 | 90% | 99.9% |
| CD105 | 68.7% | 75.9% |
| HLA-ABC | 75% | 96.9% |
| HLA-DR | Negative | Negative |
| SSEA-4 | Negative | Negative |

Method 3: Derivation of MSCs from Human ESCs Grown in mTeSR™ Media.

H9 hESCs from WiCell Research Institute, Inc. were used to derive MSCs. These cells were cultured on basement membrane matrix surface (e.g., Matrigel®, laminin, collagen, glycosaminoglycans, osteocalcin, osteonectin and mixtures thereof in mTeSR™ medium (StemCell Technologies). The hESCs were free of any form of feeder medium, conditioned media (MEF-CM/bFGF) or MEFs. Cells were checked by flow cytometry using SSEA-4-APC and CD73-PE surface antibodies to verify that the starting population of cells were ESCs (i.e., SSEA-4 positive and CD73 negative).

For differentiation, the hESCs were passaged onto new Matrigel®-coated plates with mTeSR™ medium, which was changed every three to five days. Under these culture conditions, an increasing percentage of cells displayed a spindle-shaped, stromal/fibroblast cell morphology after two to three passages. Thus, differentiation of hESCs toward MSCs was influenced by the interval of medium culture changes.

When the cultured cells on the matrix surface in the mTeSR™ medium were at least 40-50% of differentiated stromal cell morphology (spindle shaped/fibroblastic) by microscope, they were passaged using trypsin onto either 0.1% gelatin-coated plates or plates without gelatin with MSC medium (α-MEM, 10% FBS, 0.1 mM non-essential amino acids and 2 mM L-glutamine), which was changed every three to five days. Medium changes every day or every other day prevented differentiation of ESCs; likewise, medium changes at longer intervals allowed the ESCs to differentiate into some other non-MSC cell type, such as endothelial cells. Cells were checked for adherence of at least 50-75% of the passaged cells to new plastic plates. Cells were more than 90% positive for CD73 (a MSC marker) and about 1% to about 2% positive for SSEA-4 (a hESC marker), and essentially all of the cells had the stromal/fibroblast cell morphology. Any further passages were performed using trypsin, gelatin-coated (or not) plates and MSC medium. In any higher passages, cells uniformly expressed MSC markers such as CD73 (typically >99% positive), and were SSEA-4 negative. Cells were maintained continuously on MSC medium for over three months and maintained their ability to differentiate, as did cells passaged after freezing and thawing. As such, hESC-derived MSCs were consistently obtained in three to six weeks with this method.

Cells were also monitored using surface antibodies SSEA4-APC and CD73-PE for total derivation of MSC cells from ESCs. Consistent with Example 2, scrapping was avoided because undifferentiated or partially differentiated hESCs did not adhere to plastic plates.

The hESC-derived MSCs were assayed by FACS, as described above, the results of which are shown in Table 3.

TABLE 3
Cell surface markers of hESC-derived MSCs.

| Marker | MSC-mTeSR |
|---|---|
| CD73 | 96.6% |
| SSEA-4 | Negative |
| CD29 | 98.9% |
| CD44 | 99.6% |
| CD54 | 18.7% |
| CD90 | 87.5% |
| CD105 | 82% |
| CD31 | Negative |
| CD38 | Negative |
| CD34 | Negative |
| CD45 | Negative |
| HLA-ABC | 52.9% |
| HLA-DR | Negative |

### Example 4

### Differentiation of MSCs into Adipogenic Cells

Methods: Duplicate plates of 1.5×10⁵ hESC-derived MSCs (prepared as described in any of the Examples above and around passages 4-8) per ml were grown in 2 ml final volume of medium containing α-MEM with 10% FBS and adipogenic supplements (1 μM dexamethasone (Sigma Aldrich; St. Louis, Mo.), 0.5 mM methyl-isobutylxanthine (Sigma Aldrich) and 10 U/ml insulin (Sigma Aldrich)). See Pittenger et al., supra. The medium was twice per week.

Cells were checked daily for the presence of fat vacuoles under a microscope. Fat vacoules first appeared after about ten to twelve days of culture.

After 2 to 4 weeks, cells from one of the culture plates were washed 3 times with PBS, fixed in 10% formalin for 15 to 20 minutes and stained for 15 minutes with fresh Oil Red-O solution (Sigma Aldrich). Id.

Cells from the second culture plate were used for RNA preparations and for RT-PCR for PPARγ. See Barber et al., supra. and Pittenger et al., supra.

### Example 5

### Differentiation of MSCs into Chondrogenic Cells

Methods: Duplicate plates of 1×10⁶ hESC-derived MSCs (prepared as described in any of the Examples above and around passages 4-8) per ml were grown in 0.5 ml of medium comprising α-MEM with 10% FBS and chondrogenic supplements (10 ng/ml TGF-β3 (R & D Systems; Minneapolis, Minn.)) and 200 μM ascorbic acid (Sigma Aldrich) as a pelleted micromass in 15 ml conical tubes. See Pittenger et al., supra. The medium was changed every three days without disturbing the dense micromass.

After three to four weeks, cells were assayed for glycosaminoglycans within cells according to a method as described in Pittenger et al. Briefly, the micromass was fixed in formalin and paraffinized. Next, cells were deparaffinized in xylene and ethanol, stained with Weigert's iron hematoxylin for four minutes and then de-stained with fresh acid alcohol. Finally, cells were stained with 0.02% aqueous fast green FDC, washed in 1% acetic acid and stained with 0.1% aqueous Safranin-O.

Size of the micromass increased over the time course of 2 to 4 weeks in 15 ml conical tubes with chondrogenic differentiation supplements, but not in the tubes with no added supplements. In a second set of conical tubes with or without the differentiation supplements, cells were washed twice with PBS and RNA was isolated for RT-PCR and DNA-amplification with collagen II primers. See Barberi et al., supra. and Pittenger et al., supra. The results of these tests were consistent with the cells exposed to the supplements having differentiated to chondroblasts.

### Example 6

### Differentiation of MSCs into Osteogenic Cells

Methods: Duplicate plates of 1.5×10⁵ hESC-derived MSCs (prepared as described in any of the Examples above and around passages 4-8) were grown in medium containing α-MEM with 10% FBS and osteogenic differentiation supplements (100 nM dexamethasone (Sigma Aldrich), 10 mM β-glycerol phosphate (Sigma Aldrich) and 0.05 mM ascorbic acid (Sigma Aldrich). See Barberi et al., supra. and Pittenger et al., supra.

After two to four weeks, one of the plates was assayed for mineral content by the von Kossa method. Jaiswal N, et al., “Osteogenic differentiation of purified, culture-expanded human mesenchymal stem cells in vitro,” J. Cell Biochem. 64:295-312 (1997), incorporated herein by reference as if set forth in its entirety. Briefly, cells were rinsed with PBS, fixed with 10% formalin, incubated with 2% silver nitrate for thirty minutes under ultraviolet light, washed with distilled water and counterstained with hemotoxylin stain.

In the second set of wells, cells were washed twice with PBS and RNA was isolated for RT-PCR and DNA-amplification with bone specific protein primers. See Barberi et al., supra. and Pittenger et al., supra. The results observed were consistent with the cells exposed to the differentiation supplements having differentiated into osteoblast cells.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

The invention claimed is:

1. A method for making mesenchymal stromal/stem cells in vitro, the method comprising the steps of:
culturing human embryonic stem cells in a culture on a basement membrane matrix surface in a conditioned medium containing basic fibroblast growth factor, the culture being substantially free of feeder cells, and the medium being renewed no more frequently than every third day and no less frequently than every fifth day until at least about 40% of the cultured cells have a differentiated stromal/fibroblast morphology; and
passaging cells having the differentiated cell morphology onto a plastic surface in a medium that supports mesenchymal stem cells until at least about 60% of the cells on the plastic surface have the stromal/fibroblast cell morphology and express mesenchymal/stromal cell markers.

2. A method as recited in claim 1, further comprising the step of passaging the cells on the matrix surface in the medium that supports mesenchymal stem cells until at least about 40% of the cultured cells have a differentiated stromal/fibroblast cell morphology, splitting the differentiated, cells onto the matrix surface when the cells are at least 60% confluent prior to passaging the cells onto the plastic surface.

3. A method as recited in claim 1, wherein the plastic surface is coated with gelatin.

4. A method as recited in claim 1, wherein at least 75% of the cells maintained on the gelatin surface have the stromal/fibroblast cell morphology and express mesenchymal stern/stromal cell markers.

5. A method as recited in claim 1, wherein at least 85% of the cells maintained on the gelatin surface have the stromal/fibroblast cell morphology and express mesenchymal stem/stromal cell markers.

6. A method as recited in claim 1, wherein at least 95% of the cells maintained on the gelatin surface have the stromal/fibroblast cell morphology and express mesenchymal stem/stromal cell markers.

7. A method as recited in claim 1, further comprising the step of removing cells lacking the differentiated cell morphology from the culture before passaging the cells having the differentiated cell morphology.

8. A method as recited in claim 7, wherein the cells lacking the differentiated cell morphology are removed from the matrix surface when at least about 40% of cells on the surface at confluence have the differentiated cell morphology.

9. A method as recited in claim 1, wherein the cells having the differentiated cell morphology express CD73, but not SSEA-4 and Oct-4.

10. A method as recited in claim 1, wherein the bFGF is at a concentration between 3 ng/ml to 8 ng/ml.

11. A method for culturing mesenchymal lineage cells, the method comprising the steps of:
culturing human embryonic stem cells in a culture on a basement membrane matrix surface in a conditioned medium containing basic fibroblast growth factor, the culture being substantially free of feeder cells, the medium being renewed no more frequently than every third day and no less frequently than every fifth day until at least about 40% of the cultured cells have a differentiated stromal/fibroblast morphology;
passaging cells having the differentiated cell morphology onto a plastic surface in a medium that supports mesenchymal stem cells until at least about 60% of the cells on the plastic surface have the stromal/fibroblast cell morphology and express mesenchymal/stromal cell markers; and
culturing cells having the differentiated cell morphology and expressing mesenchymal/stromal cell markers under differentiating conditions to produce at least one cell type selected from the group consisting of adipocytes, chondroblasts and osteoblasts.

12. A method as recited in claim 11, further comprising the step of passaging the cells on the matrix surface in the medium that supports mesenchymal stem cells until at least about 40% of the cultured cells have a differentiated stromal/fibroblast cell morphology, splitting the cells onto the matrix surface when the cells are at least 60% confluent prior to passaging the cells onto the plastic surface.

13. A method as recited in claim 11, wherein the plastic surface is coated with gelatin.

14. A method as recited in claim 11, wherein at least 75% of the cells maintained on the gelatin surface have the stromal/fibroblast cell morphology and express mesenchymal stem/stromal cell markers.

15. A method as recited in claim 11, wherein at least 85% of the cells maintained on the gelatin surface have the stromal/fibroblast cell morphology and express mesenchymal stem/stromal cell markers.

16. A method as recited in claim 11, wherein at least 95% of the cells maintained on the gelatin surface have the stromal/fibroblast cell morphology and express mesenchymal stem/stromal cell markers.

17. A method as recited in claim 11, further comprising the step of removing cells lacking the differentiated cell morphology from the culture before passaging the cells having the differentiated cell morphology.

18. A method as recited in claim 17, wherein the cells lacking the differentiated cell morphology are removed from the matrix surface when at least about 40% of cells on the surface at confluence have the differentiated cell morphology.

19. A method as recited in claim 11, wherein the cells having the differentiated cell morphology express CD73, but not SSEA-4 and Oct-4.

20. A method as recited in claim 11, wherein the bFGF is at a concentration between 3 ng/ml to 8 ng/ml.

21. A method for making mesenchymal stromal/stem cells in vitro, the method comprising the steps of:
culturing human embryonic stem cells in a culture on a basement membrane matrix surface in a complete, serum-free medium containing basic fibroblast growth factor, the culture being free of feeder cells, and the medium being renewed no more frequently than every third day and no less frequently than every fifth day until at least about 40% of the cultured cells have a differentiated stromal/fibroblast morphology; and
passaging cells having the differentiated cell morphology onto a plastic surface in a medium that supports mesenchymal stem cells until at least about 60% of the cells on the gelatin surface have the stromal/fibroblast cell morphology and express mesenchymal/stromal cell markers.

22. A method as recited in claim 21, wherein the complete, serum-free medium is mTeSR™.

23. A method as recited in claim 21, wherein the plastic surface is coated with gelatin.

24. A method as recited in claim 21, wherein at least 75% of the cells maintained on the gelatin surface have the stromal/fibroblast cell morphology and express mesenchymal stem/stromal cell markers.

25. A method as recited in claim 21, wherein at least 85% of the cells maintained on the gelatin surface have the stromal/fibroblast cell morphology and express mesenchymal stem/stromal cell markers.

26. A method as recited in claim 21, wherein at least 95% of the cells maintained on the gelatin surface have the stromal/fibroblast cell morphology and express mesenchymal stem/stromal cell markers.

27. A method as recited in claim 21, wherein the cells having the differentiated cell morphology express CD73, but not SSEA-4 and Oct-4.
